(19) United States
(12) Patent Application Publication
(10) Pub. No.: US 2013/0023690 A1
(43) Pub. Date: Jan. 24, 2013

(54) PROCESS FOR THE PRODUCTION OF NITRILE COMPOUNDS FROM ETHYLENICALLY UNSATURATED COMPOUNDS

(71) Applicants: Sergio Mastroianni, Lyon (FR); Paul Pringle, Bristol (GB); Michael Garland, Bradford-On-Avon Wiltshire (GB); Jonathan Hopewell, Chapelthorpe (GB)

(72) Inventors: Sergio Mastroianni, Lyon (FR); Paul Pringle, Bristol (GB); Michael Garland, Bradford-On-Avon Wiltshire (GB); Jonathan Hopewell, Chapelthorpe (GB)

(21) Appl. No.: 13/378,865

(22) PCT Filed: Jun. 7, 2010
(86) PCT No.: PCT/EP10/57922

§ 371 Date: Apr. 6, 2012

(30) Foreign Application Priority Data
Jun. 16, 2009 (FR) 0954015

(51) Int. Cl. C07C 253/10

(52) U.S. Cl. 558/335

(57) ABSTRACT

A method is described for the hydrocyanation of organic ethylene-unsaturated compounds into compounds including at least one nitrile function. Also described, is a method for the hydrocyanation of a hydrocarbon compound including at least one ethylenic unsaturation by reaction in a liquid medium with hydrogen cyanide in the presence of a catalyst including a metal element selected from among the transition metals and an organophosphorous ligand including, in one embodiment of the invention, an organophosphorous compound. The described method can be used in particular for the synthesis of adiponitrile from butadiene.

The present invention relates to a process for the hydrocyanation of ethylenically unsaturated organic compounds so as to give compounds comprising at least one nitrile function.

The present invention provides a process for the hydrocyanation of a hydrocarbon-based compound comprising at least one ethylenic unsaturation, by reaction in a liquid medium with hydrogen cyanide in the presence of a catalyst comprising a metal element chosen from transition metals and an organophosphorus ligand comprising, in one embodiment of the invention, an organophosphorus compound having the following formula:

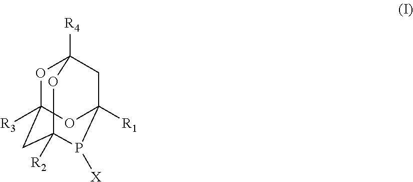

in which:
R₁, R₂, R₃ and R₄, which may be identical or different, represent a hydrogen atom, a linear or branched alkyl radical containing from 1 to 12 carbon atoms that may contain heteroatoms, a radical comprising a substituted or unsubstituted aromatic or cycloaliphatic radical that may comprise heteroatoms, a carbonyl, alkoxycarbonyl or alkoxy radical, a halogen atom, a nitrile group or a haloalkyl group containing from 1 to 12 carbon atoms,
X represents a halogen atom selected from the group consisting of fluorine and bromine.

The present invention is in particular of use for the synthesis of adiponitrile from butadiene.

The present invention relates to a process for hydrocyanation of ethylenically unsaturated organic compounds so as to give compounds comprising at least one nitrile function.

It relates more particularly to the hydrocyanation of diolefins such as butadiene or substituted olefins such as alkenenitriles, for instance pentenenitriles.

French patent No. 1 599 764 describes a process for preparing nitriles by adding hydrocyanic acid to organic compounds having at least one ethylenic double bond, in the presence of a catalyst comprising nickel and an organophosphorus ligand, a triarylphosphite. This reaction can be carried out in the presence or absence of a solvent.

When a solvent is used, it is preferably a hydrocarbon, such as benzene or xylenes, or a nitrile such as acetonitrile.

The catalyst used is an organic nickel complex, containing ligands such as phosphines, arsines, stibines, phosphites, arsenites or antimonites.

The presence of a promoter for activating the catalyst, such as a boron compound or a metal salt, generally a Lewis acid, is also recommended in said patent.

Many other catalytic systems have been proposed, generally comprising organophosphorus compounds belonging to the phosphite, phosphonite, phosphinite and phosphine family. These organophosphorus compounds can comprise one phosphorus atom per molecule and are described as monodentate ligands. They can comprise several phosphorus atoms per molecule, they are then called pluridentate ligands; more particularly, many ligands containing two phosphorus atoms per molecule (bidentate ligand) have been described in many patents.

However, the search for new catalytic systems that are more effective both in terms of catalytic activity and in terms of stability is still being undertaken.

One of the objectives of the present invention is to provide a novel family of ligands which makes it possible to obtain, with transition metals, catalytic systems which exhibit good catalytic activity in the hydrocyanation reaction.

To this effect, the present invention provides a process for the hydrocyanation of a hydrocarbon-based compound comprising at least one ethylenic unsaturation, by reaction in a liquid medium with hydrogen cyanide in the presence of a catalyst comprising a metal element chosen from transition metals and one or more organophosphorus ligands, characterized in that the organophosphorus ligand comprises at least one compound corresponding to general formula (I) or (II):

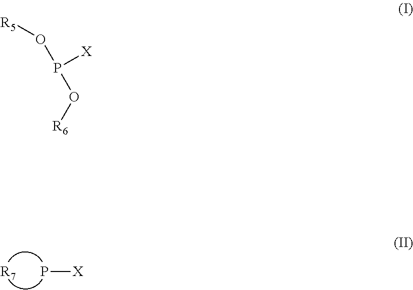

in which:
R₅ and R₆, which may be identical or different, represent a linear or branched, aliphatic monovalent radical, a monovalent radical comprising an aromatic or cycloaliphatic ring, which is substituted or unsubstituted, or several aromatic rings which are condensed or connected to one another by a covalent bond,
R₇ represents a divalent radical of general formula (III) below:

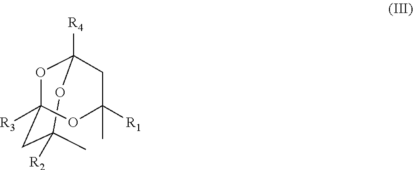

or a divalent radical of formula —(O)—R₈—(O)—, in which R₈ represents a linear or branched, aliphatic divalent radical, a divalent radical comprising an aromatic or cycloaliphatic ring, which is substituted or unsubstituted, or several aromatic rings which are condensed or connected to one another by a covalent bond,
or a divalent radical of general formula (IV) below:

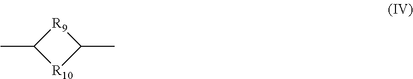

in which R₉ and R₁₀, which may be identical or different, represent a linear or branched, aliphatic divalent radical containing from 1 to 6 carbon atoms,
R₁, R₂, R₃ and R₄, which may be identical or different, represent a hydrogen atom, a linear or branched alkyl radical containing from 1 to 12 carbon atoms that may contain heteroatoms, a radical comprising a substituted or unsubstituted aromatic or cycloaliphatic radical that may comprise heteroatoms, a carbonyl, alkoxycarbonyl or alkoxy radical, a halogen atom, a nitrile group or a haloalkyl group containing from 1 to 12 carbon atoms,
X represents a halogen atom selected from the group consisting of fluorine and bromine.

Advantageously, R₁, R₂, R₃ and R₄, which may be identical or different, represent a hydrogen atom, or a linear or branched alkyl radical containing from 1 to 12 carbon atoms that may contain heteroatoms.

Preferably, the phosphorus ligand is a compound of general formula (II) according to which R₇ represents a divalent radical of general formula (III) or (IV).

Advantageously, the organophosphorus ligand is a compound corresponding to general formula (II) with the radical X representing fluorine and the radical R₇ corresponding to formula (III) or (IV).

The preferred ligands of the invention correspond to the following chemical formulae:

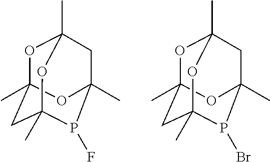

These compounds and the method for producing them have been described in several scientific communications or publications. By way of example, mention may be made of the publication by Downing et al., Organometallics, 2008, vol. 27 No. 13, pages 3216-3224.

Other preferred ligands of the invention correspond to the following chemical formulae:

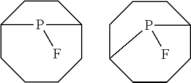

The organophosphorus ligands (fluorophosphites) corresponding to formula (I) or (II) that are suitable for the invention are in particular described in patent application US20080081759.

According to the invention, the composition of the catalytic system may be represented by general formula (V) (this formula does not correspond to the structure of the compounds and complexes present in the catalytic system):

M[L_{f}]ₜ (V)

in which:
M is a transition metal,
L_{f} represents at least one organophosphorus ligand of formula (I) or (II),
t represents a number between 1 and 10 (limits included).

In one embodiment of the invention, the ligand L_{f} is a mixture of organophosphorus compounds, at least one of which is a compound corresponding to either of general formulae (I) and (II). The mixture may comprise, for example, a monodentate organophosphite compound such as tritolyl phosphite (TTP) or triphenyl phosphite (TPP).

In the rest of the description, the term “organophosphorus compound” denotes equally the compounds of formula (I) or (II) and a mixture of organophosphorus compounds comprising, for example, an organophosphite monodentate compound and at least one compound of formula (I) or (II).

The metals M which can be complexed are, in general, any of the transition metals of groups 1b, 2b, 3b, 4b, 5b, 6b, 7b and 8 of the Periodic Table of Elements, as published in “Handbook of Chemistry and Physics, 51st Edition (1970-1971)” from The Chemical Rubber Company.

Among these metals, mention may more particularly be made of the metals that can be used as catalysts in hydrocyanation reactions. Thus, by way of nonlimiting examples, mention may be made of nickel, cobalt, iron, ruthenium, rhodium, palladium, osmium, iridium, platinum, copper, silver, gold, zinc, cadmium and mercury. Nickel is the preferred element for the hydrocyanation of unsaturated nitriles and olefins.

The preparation of the catalytic systems comprising organophosphorus compounds according to the invention can be carried out by bringing a solution of a compound of the chosen metal, for example nickel, into contact with a solution of the organophosphorus compound of the invention.

The compound of the metal can be dissolved in a solvent. The metal may be, in the compound used, either in the oxidation state that it will have in the organometallic complex or in a higher oxidation state.

By way of example, it may indicated that, in the organometallic complexes of the invention, rhodium is in the oxidation state (I), ruthenium in the oxidation state (II), platinum in the oxidation stage (0), palladium in the oxidation state (0), osmium in the oxidation state (II), iridium in the oxidation state (I), and nickel in the oxidation state (0).

If, during the preparation of the organometallic complex, the metal is used at a higher oxidation state, it may be reduced in situ.

Among the compounds of metals M that can be used for the preparation of the organometallic complexes, mention may be made, by way of nonlimiting examples, of the following nickel compounds:
compounds in which the nickel is in the zero oxidation state, such as potassium tetracyanonickelate K₄[Ni(CN)₄], bis(acrylonitrile)nickel(0), bis(1,5-cyclo-octadiene)nickel (also called Ni(cod)₂) and the derivatives containing ligands such as tetrakis(triphenylphosphine)nickel(0),
nickel compounds, such as carboxylates (in particular the acetate), carbonate, bicarbonate, borate, bromide, chloride, citrate, thiocyanate, cyanide, formate, hydroxide, hydrophosphite, phosphite, phosphate and derivatives, iodide, nitrate, sulphate, sulphite, arylsulphonates and alkylsulphonates.

When the nickel compound used corresponds to an oxidation state of the nickel of greater than 0, a reducing agent for the nickel is added to the reaction medium, which reducing agent preferably reacts with the nickel under the conditions of the reaction. This reducing agent can be organic or inorganic. Mention may be made, as nonlimiting examples, of borohydrides such as NaBH₄ or KBH₄, Zn powder, magnesium or hydrogen.

When the nickel compound used corresponds to the 0 oxidation state of nickel, a reducing agent of the type of those mentioned above can also be added, but this addition is not essential.

When an iron compound is used, the same reducing agents are suitable. In the case of palladium, the reducing agents can also be components of the reaction medium (phosphine, solvent, olefin).

The organic compounds comprising at least one ethylenic double bond more particularly used in the present process are diolefins, such as butadiene, isoprene, 1,5-hexadiene, 1,5-cyclooctadiene, ethylenically unsaturated aliphatic nitriles, particularly linear pentenenitriles, for instance 3-pentenenitrile or 4-pentenenitrile, monoolefins, for instance styrene, methylstyrene, vinylnaphthalene, cyclohexene or methylcyclohexene, and the mixtures of several of these compounds.

The pentenenitriles may comprise, in addition to the 3-pentenenitrile and the 4-pentene-nitrile, generally minor amounts of other compounds, such as 2-methyl-3-butenenitrile, 2-methyl-2-butenenitrile, 2-pentenenitrile, valeronitrile, adiponitrile, 2-methylglutaronitrile, 2-ethylsuccinonitrile or butadiene, originating, for example, from the prior reaction for the hydrocyanation of butadiene so as to give unsaturated nitriles.

This is because, during the hydrocyanation of butadiene, not insignificant amounts of 2-methyl-3-butenenitrile and 2-methyl-2-butenenitrile were formed with the linear pentenenitriles.

The catalytic system used for the hydrocyanation according to the process of the invention can be prepared before its introduction into the reaction region, for example by addition, to the organophosphorus compound(s), alone or dissolved in a solvent, the appropriate amount of chosen transition metal compound and, optionally, of reducing agent. It is also possible to prepare the catalytic system “in situ” by simple addition of the organophosphorus compound(s) and of the transition metal compound to the hydrocyanation reaction medium before or after the addition of the compound to be hydrocyanated.

The amount of compound of nickel or of another transition metal used is chosen in order to obtain a concentration, as mole of transition metal per mole of organic compounds to be hydrocyanated or isomerized, of between 10⁻⁴ and 1, and preferably between 0.005 and 0.5 mol of nickel or of the other transition metal used.

The amount of organophosphorus compounds used for forming the catalyst is chosen such that the number of moles of this compound with respect to 1 mol of transition metal is from 0.5 to 100, and preferably from 2 to 50.

Although the reaction is generally carried out without a solvent, it can be advantageous to add an inert organic solvent. The solvent may be a solvent for the catalyst which is miscible with the phase comprising the compound to be hydrocyanated at the hydrocyanation temperature. By way of examples of such solvents, mention may be made of aromatic, aliphatic or cycloaliphatic hydrocarbons.

The hydrocyanation reaction is generally carried out at a temperature of from 10° C. to 200° C., and preferably from 30° C. to 120° C. It can be carried out in a single-phase medium.

The process of the invention can be carried out continuously or batchwise.

The hydrogen cyanide used can be prepared from metal cyanides, in particular sodium cyanide, or cyanohydrins, such as acetone cyanohydrin, or by any other known synthesis process, such as the Andrussov process which consists in reacting methane with ammonia and air.

The hydrogen cyanide, free of water, is introduced into the reactor in the gaseous form or in the liquid form. It can also be dissolved beforehand in an organic solvent.

In the context of a batchwise implementation, it is in practice possible to charge to a reactor, flushed beforehand using an inert gas (such as nitrogen or argon), either a solution containing all or a portion of the various constituents, such as the organophosphorus compounds in accordance with the invention, the transition metal (nickel) compound, the optional reducing agents and solvent, or said constituents separately. Generally, the reactor is then brought to the chosen temperature and then the compound to be hydrocyanated is introduced. The hydrogen cyanide is then itself introduced, preferably continuously and unvaryingly.

When the reaction (the progress of which can be monitored by the assaying of withdrawn samples) is complete, the reaction mixture is withdrawn after cooling and the reaction products are isolated and separated, for example, by distillation.

Advantageously, the synthesis of dinitriles, such as adiponitrile, from diolefins (butadiene) is obtained in two successive stages. The first stage consists in hydrocyanating a double bond of the diolefin so as to obtain an unsaturated mononitrile. The second stage consists in hydrocyanating the unsaturation of the mononitrile so as to obtain the corresponding dinitrile(s). These two stages are generally carried out with a catalytic system comprising an organometallic complex of the same nature. However, the ratios of organophosphorus compound/metal element and concentration of the catalyst can be different. In addition, it is preferable to combine a cocatalyst or promoter with the catalytic system in the second stage. This cocatalyst or promoter is generally a Lewis acid.

The Lewis acid used as cocatalyst makes it possible, in particular, in the case of the hydrocyanation of ethylenically unsaturated aliphatic nitriles, to improve the linearity of the dinitriles obtained, i.e. the percentage of linear dinitrile relative to all the dinitriles formed, and/or to increase the activity and the lifetime of the catalyst.

The term “Lewis acid” is intended to mean, in the present text, according to the usual definition, compounds which accept electron pairs.

It is possible in particular to use the Lewis acids mentioned in the work edited by G. A. Olah “Friedel-Crafts and related Reactions”, volume I, pages 191 to 197 (1963).

The Lewis acids which can be used as cocatalysts in the present process are chosen from the compounds of elements from groups Ib, IIb, IIIa, IIIb, IVa, IVb, Va, Vb, VIb, VIIb and VIII of the Periodic Table of Elements. These compounds are most commonly salts, in particular halides, such as chlorides or bromides, sulphates, sulphonates, halosulphonates, perhaloalkyisulphonates, in particular fluoroalkylsulphonates or perfluoroalkylsulphonates, carboxylates and phosphates.

By way of nonlimiting examples of such Lewis acids, mention may be made of zinc chloride, zinc bromide, zinc iodide, manganese chloride, manganese bromide, cadmium chloride, cadmium bromide, stannous chloride, stannous bromide, stannous sulphate, stannous tartrate, indium trifluoromethylsulphonate, the chlorides or bromides of rare-earth elements, such as lanthanum, cerium, praseodymium, neodymium, samarium, europium, gadolinium, terbium, dysprosium, hafnium, erbium, thallium, ytterbium and lutetium, cobalt chloride, ferrous chloride or yttrium chloride.

Use may also be made, as Lewis acid, of organometallic compounds such as triphenylborane, titanium isopropoxide or the compounds described in the unpublished French patent applications filed on 25 Jan. 2008 under No. 08 00381 and 21 Oct. 2008 under No. 08 05821.

It is of course possible to use mixtures of several Lewis acids, as is described in the unpublished French patent application filed on 29 Jan. 2009 under No. 09 50559.

Among the Lewis acids, preference is most particularly given to zinc chloride, zinc bromide, stannous chloride, stannous bromide, triphenylborane and zinc chloride/stannous chloride mixtures, diphenylborinic anhydride and tetraisobutyl dialuminoxane.

The Lewis acid cocatalyst used generally represents from 0.01 to 50 mol per mole of transition metal compound, more particularly of nickel compound, and preferably from 1 to 10 mol per mole.

The unsaturated mononitriles used in this second stage are advantageously linear pentenenitriles such as 3-pentenenitrile, 4-pentenenitrile and mixtures thereof.

These pentenenitriles may contain generally minor amounts of other compounds, such as 2-methyl-3-butenenitrile, 2-methyl-2-butenenitrile or 2-pentenenitrile.

The catalytic solution used for the hydrocyanation in the presence of a Lewis acid can be prepared before its introduction into the reaction region, for example by addition, to the organophosphorus compounds, of the appropriate amount of chosen transition metal compound, of the Lewis acid and, optionally, of the reducing agent. It is also possible to prepare the catalytic solution “in situ” by simple addition of these various constituents to the reaction medium.

It is also possible, under the conditions of the hydrocyanation process of the present invention, and in particular by carrying out the hydrocyanation in the presence of the catalyst described above comprising at least one organophosphorus compound in accordance with the invention and at least one transition metal compound, to carry out, in the absence of hydrogen cyanide, the isomerization of 2-methyl-3-butenenitrile so as to give pentenenitriles, and more generally of branched unsaturated nitriles so as to give linear unsaturated nitriles.

The 2-methyl-3-butenenitrile subjected to isomerization according to the invention may be used alone or as a mixture with other compounds. Thus, 2-methyl-3-butenenitrile can be used as a mixture with 2-methyl-2-butenenitrile, 4-pentenenitrile, 3-pentenenitrile, 2-pentenenitrile or butadiene.

It is particularly advantageous to treat the reaction mixture originating from the hydrocyanation of butadiene with hydrocyanic acid in the presence of at least one organophosphorus compound in accordance with the invention and at least one transition metal compound, more preferably a compound of nickel in the 0 oxidation state, as defined above.

In the context of this preferred variant, since the catalytic system is already present for the reaction for the hydrocyanation of butadiene, it is sufficient to halt any introduction of hydrogen cyanide to allow the isomerization reaction to take place.

In this variant, it is possible, if appropriate, to carry out a slight flushing of the reactor using an inert gas, such as nitrogen or argon, for example, in order to drive off the hydrocyanic acid which might still be present.

The isomerization reaction is generally carried out at a temperature of between 10° C. and 200° C., and preferably between 60° C. and 140° C.

In the preferred case of an isomerization immediately following the reaction for the hydrocyanation of butadiene, it will be advantageous to carry out the isomerization at the temperature at which the hydrocyanation was carried out, or slightly above.

As for the process for the hydrocyanation of ethylenically unsaturated compounds, the catalytic system used for the isomerization can be prepared before its introduction into the reaction region, for example by mixing of the organophosphorus compound(s), of the appropriate amount of chosen transition metal compound and, optionally, of the reducing agent. It is also possible to prepare the catalytic system “in situ” by simple addition of these various constituents to the reaction medium. The amount of transition metal compound and more particularly of nickel used, and also the amount of organophosphorus compound are the same as for the hydrocyanation reaction.

Although the isomerization reaction is generally carried out without a solvent, it can be advantageous to add an inert organic solvent which may be subsequently used as extraction solvent. This is in particular the case when such a solvent has been used in the reaction for the hydrocyanation of butadiene having been used to prepare the medium subjected to the isomerization reaction. Such solvents can be chosen from those which were mentioned above for the hydrocyanation.

However, the preparation of dinitrile compounds by hydrocyanation of an olefin such as butadiene can be carried out by using a catalytic system in accordance with the invention for the stages of formation of the unsaturated nitriles and the stage of isomerization above, it being possible for the reaction for the hydrocyanation of the unsaturated nitriles so as to give dinitriles to be carried out with a catalytic system in accordance with the invention or any other catalytic system already known for this reaction.

Similarly, the reaction for the hydrocyanation of the olefin so as to give unsaturated nitriles and the isomerization of the latter can be carried out with a catalytic system different from that of the invention, the stage of hydrocyanation of the unsaturated nitriles so as to give dinitriles being carried out with a catalytic system in accordance with the invention.

Other details and advantages of the invention will be illustrated by the examples given below only by way of nonlimiting indication.

### EXAMPLES

Abbreviations used
Cod: cyclooctadiene
Ni(Cod)₂: bis(1,5-cyclooctadiene)nickel
3PN: 3-pentenenitrile
AdN: adiponitrile
ESN: ethylsuccinonitrile
MGN: methylglutaronitrile
DN: dinitrile compounds (AdN, MGN or ESN)
TTP: tritolyl phosphite
TIBAO: tetraisobutyldialuminoxane
RY(DN): real yield of dinitriles corresponding to the ratio of the number of moles of dinitriles formed to the number of moles of 3PN charged
Linearity (L): ratio of the number of moles of AdN formed to the number of moles of dinitriles formed (sum of the moles of AdN, ESN and MGN)

The following compounds: 3PN, Ni(Cod)₂, ZnCl₂, TiBAO, TTP, diphenylborinic anhydride (Ph₂BOPh₂), are known products that are commercially available.

Synthesis of the Compounds of General Formula (II):

A compound, called CgPH, having the following formula:

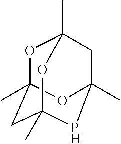

is synthesized according to the process described in the publication by Downing et al., Organometallics, 2008, vol. 27 No. 13, pages 3216-3224. This compound is used as starting material for the synthesis of the ligands A and B having the following formula:

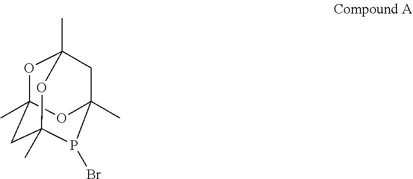

A solution of Br₂ (3.5158 g, 0.022 mol) in CH₂Cl₂ (30 ml) is added, over 30 minutes, to a solution of compound CgPH (4.3243 g, 0.02 mol) in CH₂Cl₂ (60 ml) at 0° C. and stirred at this temperature for 30 minutes, and then for one hour at ambient temperature. The solvent is evaporated off and a slightly yellow solid is obtained (Compound A). ³¹P NMR δ 53.5 (in CH₂Cl₂).

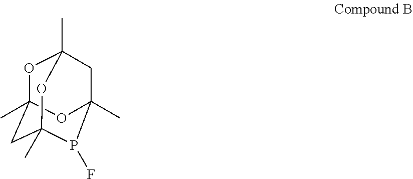

0.92 g of compound A (3.1 mmol) is added to a suspension of dry CsF (2.45 g; 16.12 mmol) in THF (50 ml) and the mixture is refluxed for 72 hours. The mixture is then filtered during cooling to ambient temperature, the solvent of the filtrate is evaporated off under vacuum and a white solid is thus obtained. 20 ml of hexane are then added, the corresponding suspension is filtered, the hexane of the organic solution is evaporated off under vacuum and a white solid is finally obtained (0.532 g, 73%) (Compound B).

Elemental analysis, found (calculated): C, 51.10 (51.28); H, 6.88 (6.89).

³¹P NMR (121 MHz; C₆D₆): δP 125.4 (d, 1J(Mp) 896.9 Hz).

¹⁹F NMR (282 MHz; C₆D₆): δF 209.84 (d, 1J(Mp) 897.1 Hz).

Two compounds, called Sym-PhobPCl and Asym-PhobPCl, having the following formulae:

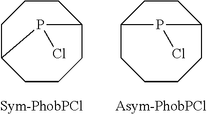

are synthesized according to the process described in the publication M. Carreira, M. Charernsuk, M. Eberhard, N. Fey, R. van Ginkel, A. Hamilton, W. P. Mul, A. G. Orpen, H. Phetmung, P. G. Pringle, J. Am. Chem. Soc, 2009, 131, 3078-3092. These compounds are used as starting material for the synthesis, respectively, of the ligands C and D having the following formulae:

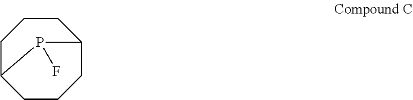

A mixture of Sym-PhobPCl (0.500 g, 2.83 mmol) and CsF (4.31 g, 28.4 mmol) in acetonitrile (8 ml) is refluxed for 1 hour. The solvent is then evaporated off and then dichloromethane (6 ml) is added. The suspension obtained is filtered and the solvent is evaporated off under vacuum.

Amount obtained: 0.341 g, 75%

Elemental analysis, found (calculated): C, 59.87 (59.99); H, 8.59 (8.81)

³¹P{¹H} NMR (CDCl₃): 159.45 (d, J_{Mp}=865 Hz)

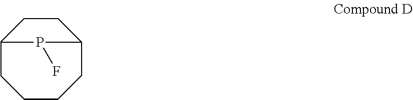

A mixture of Asym-PhobPCl (0.500 g, 2.83 mmol) and CsF (4.31 g, 28.4 mmol) in acetonitrile (8 ml) is refluxed for 1 hour. The solvent is then evaporated off and then dichloromethane (6 ml) is added. The suspension obtained is filtered and the solvent is evaporated off under vacuum.

Amount obtained: 0.193 g, 43%

³¹P{¹H} NMR (CDCl₃): 217.33 (d, J_{Mp}=808 Hz)

Compound E (2,2′-ethylidenebis(4,6-di-tert-butylphenyl)fluorophosphite) having the following formula:

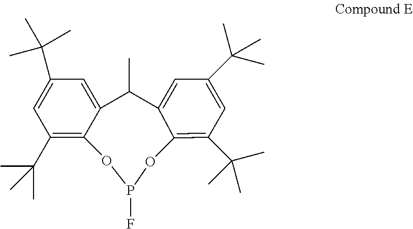 [SMILES: CC1c2cc(C(C)(C)C)cc(C(C)(C)C)c2OP(F)Oc2c1cc(C(C)(C)C)cc2C(C)(C)C]

is commercially available.

### EXAMPLES 1 to 11

### Hydrocyanation of 3-PN so as to Give AdN

The general procedure used is the following:

A 60 ml Schott-type glass tube equipped with a septum stopper is successively charged, under an argon atmosphere, with:
the ligand (ligand A, ligand B, ligand C, ligand D or ligand E) (1 mmol, 2 equivalents with respect to P)
1.21 g (15 mmol, 30 equivalents) of anhydrous 3PN
138 mg (0.5 mmol, 1 equivalent) of Ni(cod)₂
Lewis acid (see Table 1 for the amount and the nature).

The mixture is brought to 70° C., with stirring. Acetone cyanohydrin is injected into the reaction medium by means of a syringe driver at a flow rate of 0.45 ml per hour. After injecting for 3 hours, the syringe driver is halted. The mixture is cooled to ambient temperature, diluted with acetone and analysed by gas chromatography.

The results are given in the following Table 1:

TABLE 1

| | | | Lewis acid/Ni | | RY |
|---|---|---|---|---|---|
| Example | Ligand | Lewis acid | (molar) | Linearity | (DN) |
| 1 | A | ZnCl₂ | 1 | 100 | 1.9 |
| 2 | A | TIBAO | 0.5 | 94.9 | 2.8 |
| 3 | A | Ph₂BOBPh₂ | 0.5 | 100 | 2.4 |
| 4 | B | ZnCl₂ | 1 | 65.6 | 82.6 |
| 5 | B | TIBAO | 0.5 | 69.3 | 31.5 |
| 6 | B | Ph₂BOBPh₂ | 0.5 | 84.9 | 20.6 |
| 7 | E | ZnCl₂ | 1 | 63.7 | 26.6 |
| 8 | E | TIBAO | 0.5 | 51.2 | 9.5 |
| 9 | E | Ph₂BOBPh₂ | 0.5 | 70.5 | 11.7 |
| 10 | C | Ph₂BOBPh₂ | 0.5 | 81.8 | 19.2 |
| 11 | D | ZnCl₂ | 1 | 100 | 1 |

### EXAMPLE 12

### Hydrocyanation of 3-PN so as to Give AdN

The general procedure used is the following:

A 60 ml Schott-type glass tube equipped with a septum stopper is successively charged, under an argon atmosphere, with:
0.32 mmol of ligand
5 mmol of anhydrous 3PN
0.17 mmol of Ni(cod)₂
0.15 mmol of ZnCl₂

The mixture is brought to 70° C., with stirring. Acetone cyanohydrin is injected into the reaction medium by means of a syringe driver at a flow rate of 0.45 ml per hour. After injecting for 3 hours, the syringe driver is halted. The mixture is cooled to ambient temperature, diluted with acetone, and analysed by gas chromatography.

The results are given in the following Table 2:

TABLE 2

| | | | Lewis acid/Ni | | RY |
|---|---|---|---|---|---|
| Example | Ligand | Lewis acid | (molar) | Linearity | (DN) |
| 12 | C | ZnCl₂ | 0.9 | 76.4 | 12.8 |

### EXAMPLES 13 and 14

### Hydrocyanation of 3-PN so as to Give AdN A 60 ml Schott-type glass tube equipped with a septum stopper is successively charged, under an argon atmosphere, with:

ligand 1 (see Table 3 for nature and amount)
ligand 2 (see Table 3 for nature and amount)
1.21 g (15 mmol, 30 equivalents) of 3PN
138 mg (0.5 mmol, 1 equivalent) of Ni(cod)₂
Lewis acid (see Table 3 for nature and amount)

The mixture is brought to 70° C., with stirring. Acetone cyanohydrin is injected into the reaction medium by means of a syringe driver at a flow rate of 0.45 ml per hour. After injecting for 3 hours, the syringe driver is halted. The mixture is cooled to ambient temperature, diluted with acetone, and analysed by gas chromatography.

The results are given in the following Table 3:

TABLE 3

| | | | | | Lewis | | |
|---|---|---|---|---|---|---|---|
| | | | Ligand1/Ligand2/Ni | | acid/Ni | | RY |
| Example | Ligand 1 | Ligand 2 | (molar equivalents) | Lewis acid | (molar) | Linearity | (DN) |
| 13 | TTP | B | 4.5/0.5/1 | Ph₂BOBPh₂ | 0.5 | 90 | 5.2 |
| 14 | TTP | — | 5/0/1 | Ph₂BOBPh₂ | 0.5 | 73.8 | 1.2 |
| comparative | | | | | | | |

1. A process for the hydrocyanation of a hydrocarbon-based compound comprising at least one ethylenic unsaturation, the process comprising reacting in a liquid medium with hydrogen cyanide in the presence of a catalyst comprising a metal element selected from the group consisting of transition metals and an organophosphorus ligand, wherein the organophosphorus ligand comprises at least one compound corresponding to general formula (I) or (II): 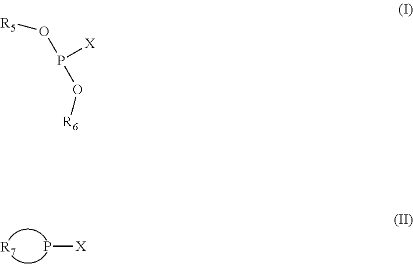
in which:
R₅ and R₆, which can be identical or different, represent a linear or branched, aliphatic monovalent radical, a monovalent radical comprising an aromatic or cycloaliphatic ring, which is substituted or unsubstituted, or several aromatic rings which are condensed or connected to one another by a covalent bond,
R₇ represents a divalent radical of general formula (III) below: 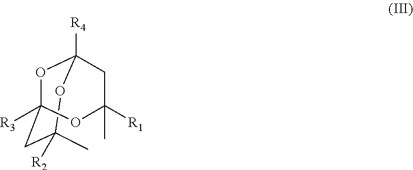
or a divalent radical of formula —O—R₈—O—, in which R₈ represents a linear or branched, aliphatic divalent radical, a divalent radical comprising an aromatic or cycloaliphatic ring, which is substituted or unsubstituted, or several aromatic rings which are condensed or connected to one another by a covalent bond,
or a divalent radical of general formula (IV) below: 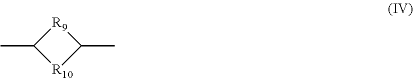
in which R₉ and R₁₀, which can be identical or different, represent a linear or branched, aliphatic divalent radical containing from 1 to 6 carbon atoms,
R₁, R₂, R₃ and R₄, which can be identical or different, represent a hydrogen atom, a linear or branched alkyl radical containing from 1 to 12 carbon atoms that can contain heteroatoms, a radical comprising a substituted or unsubstituted aromatic or cycloaliphatic radical which can comprise heteroatoms, a carbonyl, alkoxycarbonyl or alkoxy radical, a halogen atom, a nitrile group or a haloalkyl group containing from 1 to 12 carbon atoms,
X represents a halogen atom selected from the group consisting of fluorine and bromine.

2. The process according to claim 1, wherein R₁, R₂, R₃ and R₄, which may can be identical or different, represent a hydrogen atom, or a linear or branched alkyl radical containing from 1 to 12 carbon atoms that can contain heteroatoms.

3. The process according to claim 1, wherein the phosphorus ligand is a compound of general formula (II) in which R₇ represents a divalent radical of general formula (III) or (IV).

4. The process according to claim 1, wherein the compound of general formula (II) corresponds to either of the following formulae:

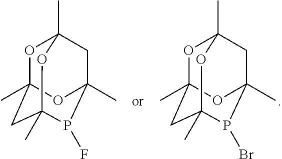

5. The process according to claim 1, wherein the compound of general formula (II) corresponds to either of the following formulae:

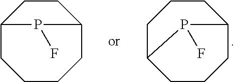

6. The process according to claim 1, wherein the metal element is selected from the group consisting of nickel, cobalt, iron, ruthenium, rhodium, palladium, osmium, iridium, platinum, copper, silver, gold, zinc, cadmium and mercury.

7. The process according to claim 1, wherein the composition of the catalytic system is expressed by general formula (V):
M[L_{f}]ₜ (V)
in which:
M is a transition metal,
L_{f} represents the organophosphorus ligand(s), at least one of which corresponds to a compound of formula (I) or (II), and
t represents a number between 1 and 10 (limits included).

8. The process according to claim 7, wherein L_{f} represents a mixture of organophosphorus ligands comprising at least one ligand corresponding to a compound of formula (I) or (II) and at least one monodentate organophosphite ligand.

9. The process according to claim 8, wherein the monodentate organophosphite ligand is selected from the group consisting of tritolyl phosphite and triphenyl phosphite.

10. The process according to claim 1, wherein the organic compounds comprising at least one ethylenic double bond are selected from the group consisting of diolefins ethylenically unsaturated aliphatic nitriles, monoolefins and also mixtures of several of these compounds.

11. The process according to claim 1, wherein the amount of compound of nickel or of another transition metal used is chosen such that there is, per mole of organic compound to be hydrocyanated or isomerized, between 10⁻⁴ and 1 mol of nickel or of the other transition metal used, and in that the amount of organophosphorus compounds used is chosen such that the number of moles of these compounds with respect to 1 mol of transition metal is from 0.5 to 100.

12. The process according to claim 1, wherein the process is conducted hydrocyanation of ethylenically unsaturated nitrile compounds so as to give dinitriles, by reaction with hydrogen cyanide, wherein the reaction is carried out in the presence of a catalytic system comprising at least one compound of a transition metal, at least one compound of formula (I) or (II) and a cocatalyst consisting of at least one Lewis acid.

13. The process according to claim 12, wherein the ethylenically unsaturated nitrile compounds are selected from the group consisting of ethylenically unsaturated aliphatic nitriles and mixtures thereof.

14. The process according to claim 12, wherein the Lewis acid used as cocatalyst is a compound of an element from groups Ib, IIb, IIIa, IIIb, IVa, IVb, Va, Vb, VIb, VIIb or VIII of the Periodic Table of Elements.

15. The process according to claim 12, wherein the Lewis acid is selected from the group consisting of zinc chloride, zinc bromide, zinc iodide, manganese chloride, manganese bromide, cadmium chloride, cadmium bromide, stannous chloride, stannous bromide, stannous sulphate, stannous tartrate, indium trifluoromethylsulphonate, the chlorides or bromides of rare-earth elements, such as lanthanum, cerium, praseodymium, neodymium, samarium, europium, gadolinium, terbium, dysprosium, hafnium, erbium, thallium, ytterbium and lutetium, cobalt chloride, ferrous chloride, yttrium chloride, and mixtures thereof, and organometallic compounds.

16. The process according to claim 1, wherein the isomerization, so as to give pentenenitriles, of the 2-methyl-3-butenenitrile present in the reaction mixture originating from the hydrocyanation of butadiene is carried out in the absence of hydrogen cyanide, the isomerization being carried out in the presence of a catalyst comprising at least one compound of formula (I) or (II) and at least one compound of a transition metal.

17. The process according to claim 10, wherein when the organic compound is diolefin, the diolefin is selected from the group consisting of butadiene, isoprene, 1,5-hexadiene and 1,5-cyclooctadiene.

18. The process according to claim 10, wherein when the organic compound is an ethylenically unsaturated aliphatic nitrile, the organic compound is a linear pentenitrile.

19. The process according to claim 18, wherein the linear pentenitrile is 3-pentenitrile or 4-pentenitrile.

20. The process according to claim 10, wherein when the organic compound is a monolefin, the monoolefin is selected from the group consisting of styrene, methylstyrene, vinylnaphthalene, cyclohexene and methylcyclohexene.

21. The process according to claim 13, wherein the ethylenically unsaturated aliphatic nitriles comprise a linear pentenitrile selected from the group consisting of 3-pentenitrile, 4-pentenitrile and mixtures thereof.
